SEQUENCE LISTING (10 sequences)

SEQ ID NO: 1 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 2 - DNA, 3020 bp, Homo sapiens
gttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagcctcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtaaaacaagtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtctggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaagaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggactgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagatattgatgataacacttaaaattgtaacctgcatttttcactttgctctcaattaaagtctattcaaaaggaaaaaaaaaaaaaaaa

SEQ ID NO: 3 - protein, 280 aa, Homo sapiens
GSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQ

SEQ ID NO: 4 - DNA, 843 bp, Homo sapiens
ggctccgacgttcgggacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcagtga

SEQ ID NO: 5 - protein, 292 aa, Homo sapiens
MNNNDLFQASRHGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQ

SEQ ID NO: 6 - DNA, 882 bp, Homo sapiens
atgaacaataacgatctgtttcaggcgagccgccacggctccgacgttcgggacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcagtgatga

SEQ ID NO: 7 - protein, 9 aa, Homo sapiens
ALLPAVPSL

SEQ ID NO: 8 - protein, 9 aa, Homo sapien
VLDFAPPGA

SEQ ID NO: 9 - protein, 429 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 10 - DNA, 1291 bp, Homo sapiens
atgggctccgacgttcgtgacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttgag

(19) United States
(12) Patent Application Publication
(10) Pub. No.: US 2005/0260217 A1
(43) Pub. Date: Nov. 24, 2005

(54) Co-encapsulated WT1 polypeptide and immunostimulant microsphere formulations and methods thereof

(75) Inventors: Mark E. Johnson, Los Altos, CA (US); Afsaneh Mozaffarian, Bellevue, WA (US); Sally P. Mossman, Seattle, WA (US); Charlie D. Meert, Newcastle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 11/071,062

(22) Filed: Mar. 3, 2005

Related U.S. Application Data
(60) Provisional application No. 60/550,362, filed on Mar. 4, 2004.

(51) Int. Cl. A61K 39/00; A61K 9/14; A61K 31/739

(52) U.S. Cl. 424/185.1; 514/54; 424/489

(57) ABSTRACT

Compositions and methods for the therapy of malignant diseases, such as leukemia and cancer, are disclosed. The compositions comprise one or more of a WT1 WT1 polypeptides in combination with one or more immunostimulants, either independently or co-encapsulated with biodegradable polymeric microspheres. Such compositions may be used, for example, for the prevention and treatment of metastatic diseases.

### TECHNICAL FIELD OF THE INVENTION

The invention relates to formulations, compositions and methods that can be used for the delivery of WT1 polypeptide or polynucleotide vaccines and immunostimulants. More particularly, the invention relates to co-encapsulated microsphere compositions that enable more efficient and effective delivery of WT polypeptide or polynucleotide vaccines and immunostimulants.

### BACKGROUND OF THE INVENTION

The present invention relates in general to compositions and methods to generate or enhance an immune response to WT1, and to the use of such compositions for preventing and/or treating malignant diseases. Cancer and leukemia are significant health problems in the United States and throughout the world. Although advances have been made in detection and treatment of such diseases, no vaccine or other universally successful method for prevention or treatment of cancer and leukemia is currently available.

The immune response raised against a Wilms Tumor (WT) gene product (e.g., WT1) can provide prophylactic and/or therapeutic benefit for patients afflicted with malignant diseases characterized by increased WT1 gene expression. Such diseases include, but are not limited to, hematopoetic proliferation disorders, including leukemias e.g., acute myeloid leukemia (AML), chronic myeloid leukemia (CML), acute lymphocytic leukemia (ALL and childhood ALL), myelodysplastic syndrome (MDS), malignant mesothelioma, as well as lymphoma, many cancers such lung, breast, ovarian, thyroid and gastrointestinal cancers and melanomas. The WT1 gene was originally identified and isolated on the basis of a cytogenetic deletion at chromosome 11p13 in patients with Wilms' tumor (see Call et al., U.S. Pat. No. 5,350,840). Further characterization of WT1 polypeptides, variants, mimetics is found in U.S. Pat. Nos. 5,350,840, 5,726,288 and 6,316,599, WIPO Published Patent Application Nos: WO 91/07509, WO 00/18795, WO 02/28414, WO 03/037060, WO 01/62920, US Published Patent Application Nos: U.S. 20030082196, U.S. 20030072767, U.S. 20030095971, U.S. 20030039635, U.S. 20030198622, U.S. 20030215458 and U.S. 20040018204.

The present invention provides WT1 polypeptides or polynucleotides encapsulated within microspheres in combination with an immunostimulant, preferably the immunostimulant is co-encapsulated with the WT1 polypeptide. An effective cancer and/or leukemia vaccine will likely elicit CTL responses in addition to T-helper responses and antibodies. Encapsulated proteins elicit strong and comprehensive immune responses, including both cellular and humoral immune responses and microspheres can encapsulate proteins of substantial size. Such a WT1 polypeptide or polynucleotide encapsulated vaccine allows for continuous boosting without compromising the immune response, unlike viral vectors. Such a vaccine is also safe due to low reactogenicity of PLG and encapsulated adjuvant. Co-encapsulation of vaccine antigen and immunostimulant allows for targeting of both the vaccine antigen and immunostimulant to same cell, resulting in increased efficiency with the added benefit of providing a product with a potential greater stability at higher temperatures (e.g. 4° C.) and can be administered as a single vial vaccine, thereby eliminating the need for admixing immunostimulant at the patient bedside.

Accordingly, there is a need in the art for improved compositions and methods for leukemia and cancer prevention and therapy. The present invention fulfills these needs and further provides other related advantages.

### BRIEF SUMMARY OF THE INVENTION

Briefly stated, this invention provides compositions and methods for the diagnosis and therapy of diseases such as leukemia and cancer. In one aspect, the present invention provides a WT1 polypeptide and an immunostimulant co-encapsulated in biodegradable polymeric microspheres in combination with a pharmaceutically acceptable carrier or excipient. Within certain embodiments the immunostimulant is an adjuvant. Preferably the adjuvant is selected from monophosphoryl lipid A, 3-de-O-acylated monophosphoryl lipid A, an aminoalkyl glucosaminide 4-phosphate compound, a saponin, aluminum phosphate, calcium phosphate, cell wall skeleton, or a CpG-containing oligonucleotide, either alone or in combination. Within one embodiment the WT1 polypeptide is a WT1 fusion polypeptide of SEQ ID NO:5. Within another embodiment the adjuvant is monophosphoryl lipid A or 3-de-O-acylated monophosphoryl lipid A.

The present invention further provides methods for enhancing or inducing an immune response in a human patient, comprising administering to a patient a pharmaceutical composition or vaccine as described above. In certain embodiments, the patient is a human.

These and other aspects of the present invention will become apparent upon reference to the following detailed descriptions.

All of the U.S. patents, U.S. patent application publications, U.S. patent applications, foreign patents, foreign patent applications and non-patent publications referred to in this specification and/or listed in the Application Data Sheet, including but not limited to U.S. Application No. 60/550,362, filed Mar. 4, 2004, are hereby incorporated by reference in their entirety as if each was incorporated individually.

### BRIEF DESCRIPTION OF THE SEQUENCE IDENTIFIERS

SEQ ID NO:1 is the amino acid sequence for the full length native human WT1 protein.

SEQ ID NO:2 is the polynucleotide sequence encoding the full length native human WT1 protein.

SEQ ID NO:3 is the amino acid sequence for the WT1 F fragment of WT1 (amino acids 2-281 of the splice variant of WT1 as set forth in SEQ ID NO:9).

SEQ ID NO:4 is the polynucleotide sequence encoding the WT1 F fragment of SEQ ID NO:3.

SEQ ID NO:5 is the amino acid sequence for the WT1 F-Truncated TAT fusion protein.

SEQ ID NO:6 is the polynucleotide sequence encoding the WT1 F-truncated TAT fusion protein of SEQ ID NO:5.

SEQ ID NO:7 is the WT-1 CD8+ T cell epitope p10-18 (ALLPAVPSL).

SEQ ID NO:8 is the WT-1 CD8+ T cell epitope p37-45 (VLDFAPPGA).

SEQ ID NO:9 is the amino acid sequence of a splice variant of human WT1.

SEQ ID NO:10 is the polynucleotide sequence of a splice variant of human WT1, encoding the amino acid sequence set forth in SEQ ID NO:9.

### DETAILED DESCRIPTION OF THE INVENTION

The invention provides compositions comprising one or more WT1 polypeptides or polynucleotides encapsulated within biodegradable polymeric microspheres and pharmaceutically acceptable carriers or excipients as well as methods of using such compositions for the immunotherapy of malignant diseases. Within one embodiment, one or more immunostimulants are administered with the encapsulated WT1 polypeptides or polynucleotides. Preferably the immunostimulants are co-encapsulated in the microspheres along with the WT1 polypeptides or polynucleotides.

The WT1 polypeptides or polynucleotides may be delivered via encapsulation in polymeric microspheres or co-encapsulation with one or more immunostimulants in the same microsphere. Encapsulated WT1 polypeptides or polynucleotide microsphere compositions may be co-administered with microspheres having one or more encapsulated immunostimulants. Immunostimulants free of microsphere formulations may also be co-administered with encapsulated WT1 polypeptide or polynucleotide microsphere compositions. Alternatively, WT1 polypeptide or polynucleotide free of microsphere formulation may be co-administered with immunostimulant microsphere compositions. Also contemplated by the present invention are WT1 polypeptides or polynucleotides and/or immunostimulants that are bound, either covalently or non-covalently, to the surface of microspheres.

WT1 Polypeptides

Protein or polypeptide as used herein refers to a polymer of at least about 8, 9, or about 10 amino acids, or more typically at least about 15, 20, 25, 30, 35, 40, 45, or about 50 amino acids. In certain embodiments, a WT1 polypeptide is of intermediate length to a full length WT1 protein (e.g., WT F, corresponding to amino acids 1-281 of SEQ ID NO:9). Such proteins or polypeptides may have primary, secondary, tertiary and, in some cases, quaternary, structures. The protein or polypeptide can be isolated from natural sources, produced by recombinant techniques or chemically synthesized.

Within the context of the present invention, a WT1 polypeptide is a polypeptide that comprises at least an immunogenic portion of a native WT1 (i.e., a WT1 protein expressed by an organism that is not genetically modified), or a variant or mimetic thereof, as described herein. A WT1 polypeptide may be of any length, provided that it comprises at least an immunogenic portion of a native protein or a variant thereof. A WT1 polypeptide may be an oligopeptide (i.e., consisting of a relatively small number of amino acid residues, such as 8-10 residues, joined by peptide bonds), a polypeptide of intermediate size, a full length WT1 protein or a WT1 fusion polypeptide. Exemplary WT1 polypeptides are disclosed in U.S. Pat. Nos. 5,350,840, 5,726,288 and 6,316,599, WIPO Published Patent Application Nos: WO 91/07509, WO 00/18795, WO 02/28414, WO 03/037060, WO 01/62920, US Published Patent Application Nos: U.S. 20030082196, U.S. 20030072767, U.S. 20030095971, U.S. 20030039635, U.S. 20030198622, U.S. 20030215458 and U.S. 20040018204. The polypeptide sequence for the full length native human WT1 protein and polynucleotide sequence encoding it are provided in SEQ ID NOs: 1 and 2. The polypeptide sequence for a splice variant of WT1 and the polynucleotide sequence encoding it are provided in SEQ ID NOs:9 and 10. An exemplary polypeptide of intermediate size (amino acid residues 2-281 of the splice variant of WT1 as set forth in SEQ ID NO:9) is provided in SEQ ID NO:3 and the polynucleotide sequence encoding it in SEQ ID NO:4. This polypeptide is referred to herein as WT1 F. As would be recognized by the skilled artisan, this fragment of WT1 can be cloned into an appropriate vector containing a codon encoding a start methionine residue (equivalent to amino acid 1 of a WT1 protein), and used to express the WT1 F protein fragment for purification and further use. In further embodiments, this fragment can be engineered to any of a variety of fusion partners as described further herein.

As used herein, an immunogenic portion is a portion of a polypeptide that is recognized (i.e., specifically bound) by a B-cell and/or T-cell surface antigen receptor. Certain preferred immunogenic portions bind to an MHC class I or class II molecule.

As used herein, an isolated polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. With regard to polynucleotides, “isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man. Thus, a polynucleotide is considered to be isolated if, for example, it is cloned into a vector that is not a part of the natural environment.

As used herein, a polypeptide variant is a polypeptide that differs from a native polypeptide in one or more substitutions, deletions, additions and/or insertions, such that the immunogenicity of the polypeptide is retained (i.e., the ability of the variant to react with antigen-specific antisera and/or T-cell lines or clones is not substantially diminished relative to the native polypeptide).

As used herein, WT1 mimetics may comprise amino acids linked to one or more amino acid mimetics (i.e., one or more amino acids within the WT1 protein may be replaced by an amino acid mimetic) or may be entirely nonpeptide mimetics. An amino acid mimetic is a compound that is conformationally similar to an amino acid such that it can be substituted for an amino acid within a WT1 polypeptide without substantially diminishing the ability to react with antigen-specific antisera and/or T cell lines or clones. A nonpeptide mimetic is a compound that does not contain amino acids, and that has an overall conformation that is similar to a WT1 polypeptide such that the ability of the mimetic to react with WT1-specific antisera and/or T cell lines or clones is not substantially diminished relative to the ability of a WT1 polypeptide.

As used herein, a WT1 fusion polypeptide is a fusion polypeptide that comprises one or multiple WT1 polypeptides as described herein, or that comprises at least one WT1 polypeptide as described herein and an unrelated fusion partner. The fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide. A preferred WT1 fusion polypeptide is the truncated twin arginine translator (TAT) signal peptide fused to a truncated WT1 polypeptide, (WT1 F amino acid residues 2-281 of SEQ ID NO:9), as described in published U.S. Patent Application Nos: U.S. 200302154458 and U.S. 20040018204 and WIPO Published Patent Application No: WO03/037060, and provided in SEQ ID NO:5, the polynucleotide sequence encoding the WT1 fusion polypeptide is provided in SEQ ID NO:6.

WT1 fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system.

WT1 polypeptides may be prepared using any of a variety of techniques known to those of ordinary skill in the art. For example, recombinant WT1 polypeptides encoded by WT1 polynucleotides, as described herein, may be readily prepared from the polynucleotide. In general, any of a variety of expression vectors known to those of ordinary skill in the art may be employed to express recombinant WT1 polypeptides. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast, baculovirus or a mammalian cell line such as COS or CHO. The WT1 polypeptides may be conjugated to a signal (or leader) sequence at the N-terminal end of the protein which co-translationally or post-translationally directs transfer of the protein. A polypeptide may also, or alternatively, be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

The recombinant WT1 polypeptides can be purified using fractionation and/or conventional purification methods or media.

WT polypeptides may also be synthesized commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc. (Foster City, Calif.), and may be operated according to the manufacturer's instructions.

WT1 Polynucleotides

Any polynucleotide that encodes a WT1 polypeptide as described herein is a WT1 polynucleotide encompassed by the present invention. Such polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

WT1 polynucleotides encode WT1 polypeptides which include oligopeptides, full length WT1 proteins, WT1 polypeptides of intermediate size, WT1 variants, minetics, or WT1 fusion polypeptides. Such WT1 polynucleotides are disclosed in U.S. Pat. Nos. 5,350,840, 5,726,288 and 6,316,599, WIPO Published Patent Application Nos: WO 91/07509, WO 00/18795, WO 02/28414, WO 03/037060, WO 01/62920, US Published Patent Application Nos: U.S. 20030082196, U.S. 20030072767, U.S. 20030095971, U.S. 20030039635, U.S. 20030198622, U.S. 20030215458 and U.S. 20040018204.

WT1 polynucleotides may be prepared using any of a variety of techniques known to those of ordinary skill in the art. For example, a WT1 polynucleotide may be amplified from cDNA prepared from cells that express WT1, using known techniques such as polymerase chain reaction (PCR).

WT1 polynucleotides may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Polynucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art. In particular, one embodiment of the invention comprises expression vectors which incorporate the nucleic acid molecules of the invention, in operable linkage (i.e., “operably linked”) to an expression control sequence (promoter). Construction of such vectors, such as viral (e.g., adenovirus or Vaccinia virus) or attenuated viral vectors is well within the skill of the art, as is the transformation or transfection of cells, to produce eukaryotic cell lines, or prokaryotic cell strains which encode the molecule of interest. Exemplary of the host cells which can be employed in this fashion are COS cells, CHO cells, yeast cells, insect cells (e.g., Spodoptera frugiperda or Sf-9 cells), NIH 3T3 cells, and so forth. Prokaryotic cells, such as E. coli and other bacteria may also be used.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art. cDNA constructs within such a vector may be used, for example, to transfect human or animal cell lines for use in establishing WT1 positive tumor models which may be used to perform tumor protection and adoptive immunotherapy experiments to demonstrate tumor or leukemia-growth inhibition or lysis of such cells.

Immunostimulants

Also included within the scope of the invention is the combination of one or more immunostimulants in addition to the WT1 polypeptides or polynucleotides. Thus, the present invention provides compositions comprising one or more WT1 polypeptides or polynucleotides in combination with one or more immunostimulants. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following the application of a vaccine, as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays, such as measuring the ration of IgG1:IgG2 in serum antibody responses. For a review of the families of cytokines, see Mosmann and Coffman, Ann. Rev. Immunol. 7:145-173, 1989.

Certain preferred adjuvants for eliciting a predominantly Th1-type response include, for example, monophosphoryl lipid A, 3-de-O-acylated monophosphoryl lipid A (MPL® adjuvant, Corixa Corporation, Seattle, Wash.), either alone or in combination with trehalose dimycolates, pryidine-soluble extract, and/or cell wall skeleton, see, for example, U.S. Pat. Nos. 4,877,611; 4,806,352, 4,803,070, 4,987,237, 4,887,611, 4,912,094, 6,491,919, 6,630,161, and WIPO Published Patent Application No. WO02/078637. Aminoalkyl glucosaminide phosphates (AGPs), synthetic mono- and disaccharide lipid A mimetics, preferably the AGP known as RC529 (B15) (Corixa Corporation, Seattle, Wash.), see, for example, U.S. Pat. Nos. 6,525,028, 6,113,918, 6,355,257, 6,303,347, and WIPO Published Patent Application No. WO 98/50399 and 02/12258; alone or in combination with other adjuvants.

CpG-containing oligonucleotides (in which the CpG dinucleotide is unmethylated) also induce a predominantly Th1 response. Such oligonucleotides are well known and are described, for example, WIPO Published Patent Applications WO 96/02555 and WO 99/33488 and U.S. Pat. Nos. 5,554,744; 5,856,462; 6,008,200; 6,194,388; 6,207,646; 6,239,116; 6,406,705; 6,426,334; 6,476,000, 6,544,518 and 6,558,670. Immunostimulatory DNA sequences are also described, for example, by Sato et al., Science 273:352, 1996.

Another preferred adjuvant comprises a saponin, such as Quil A, or derivatives thereof, including QS21 and QS7 (Antigenics, New York, N.Y.) see U.S. Pat. Nos. 5,057,540, 5,273,965, 5,443,829, 5,583,112, 5,650,398, 6,231,859, and 6,524,584; Escin (Aescigenin-(2-methyl-3-acetoxybutyrate)-(2-xylosido4-glucosidoglucuronoside); Digitonin; Gypsophila or Chenopodium quinoa saponins or formulations that include combinations of two or more saponins. Also included is a less reactogenic composition where the QS21 is quenched with cholesterol, as described in WIPO Published Patent Application No. WO 96/33739. Within other embodiments adjuvant compositions include the combination of monophosphoryl lipid A, 3-de-O-acylated monophosphoryl lipid A or AGP with one or more saponins, such as the combination of QS21 and MPL® adjuvant or QS21 and RC529, see, for example, WIPO Published Patent Application Nos.: WO 94/00153, WO 95/17210 and WO 01/78777. Also included are combinations of CpG-containing oligonucleotides and saponin derivatives, such as the combination of CpG and QS21, see, for example, WIPO Published Patent Application No. WO 00/09159.

Other adjuvants include Freund's Incomplete Adjuvant and Complete Adjuvant (Difco Laboratories, Detroit, Mich.), Merck Adjuvant 65 (Merck and Company, Inc., Rahway, N.J.), Montanide ISA 720 (Seppic, Paris, France), SAF (Chiron, Emeryville, Calif.), ISCOMS (CSL, Parkeville, Victoria, Australia), MF-59 (Chiron, Emeryville, Calif.), the SBAS series of adjuvants (e.g., SBAS-2 or SBAS-4, SmithKline Beecham, Rixensart, Belgium), polyoxyethylene ether adjuvants such as those described in WIPO Published Patent Application No. WO 99/52549A1, isotucerosol, see WIPO Published Patent Application No: WO 01/70663, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides and polyphosphazenes. Cytokines, such as GM-CSF, interleukin-2, -7, -12, and other like growth factors, may also be used as adjuvants.

Encapsulation in Microspheres

Encapsulation of protein and/or adjuvant can be achieved by a variety of methods known in the art (U.S. Pat. Nos. 5,407,609 and 4,897,268; WIPO Published Patent Application Nos: WO02/03961 and WO02/092132). The encapsulation is preferably performed via hydrophobic ion pairing as described in WIPO Published Patent Application No. WO 03/005952. Briefly, hydrophobic ion pairing (HIP) involves stoichiometric replacement of polar counter ions with a species of similar charge but less easily solvated in water. This provides a method to change the solubility properties of proteins, allowing solubilization of the protein into an organic solvent, such as dimethyl sulfoxide. The HIP method may comprise extracting an aqueous solution comprising the protein, in this case one or more WT1 polypeptides, with an organic solvent containing a hydrophobic ion pairing (HIP) agent, or precipitating the protein with an HIP agent and then dissolving the precipitated complex in an organic solvent.

The HIP agent may be an anionic HIP agent, such as docusate sodium, sodium dodecyl sulfate, or sodium oleate. The HIP agent may also be a cationic HIP agent, such as dimediyldioctadecyl-ammonium bromide (DDAB18); 1,2 dioleoyloxy-3-(trimethyl-ammonium) propane (DOTAP); or cetrimonium bromide (CTAB). The HIP agent and the aqueous solution are selected in accordance with the characteristics of the protein to be encapsulated. Typically, for proteins having an isoelectric point (pI) at or below 7.0, an anionic HIP agent is preferred. Likewise, for proteins having a pI greater than or equal to 7.0, a cationic HIP agent is preferred. For encapsulation of a protein having a pI of about 7.0, either a cationic or anionic HIP agent can be used. The pH of the aqueous solution can be adjusted to achieve the appropriate charge characteristics (preferably at least two pH units above or below the pI of the protein). Preferably for HIP extraction, the organic medium has a ratio of HIP agent to protein of up to about 70:1; the organic phase can then be recovered from the extraction by centrifugation (salt and HIP agent concentrations can be optimized to obtain cleaner phase separation upon centrifugation). Typically, aqueous solutions having low salt concentrations are preferred, typically the addition of a low concentration of a salt such as 4 mM calcium chloride is preferred. In one embodiment, the aqueous solution has a total salt concentration of less than about 30 mM. Preferably for HIP precipitation, the HIP agent is present in stoichiometric amounts equal to or greater than the number of opposite charges on the protein at the pH of choice; the precipitated complex can then be solubilized in an organic solvent. Following extraction or precipitation, a wall-forming material, and optionally immunostimulants, or other additives that are soluble in an organic solvent, such as methylene chloride, may then be dissolved in the organic phase with the protein.

In one embodiment, the HIP agent is docusate sodium and it is used at approximately 2 times molar excess the positive charges on the WT1 polypeptide at pH 3 (that is, approximately 2.3 mg docusate sodium per 3 mg WT1) to precipitate the protein out of an aqueous phase at pH 3 into a precipitated protein-docusate sodium complex. As would be understood by the skilled arisan, these conditions can be optimized depending on the specific protein. The precipitate is then isolated and solubilized in dimethyl sulfoxide. The protein containing DMSO solution is then preferably combined with a second solution containing PLG RG502H, MPL, and methylene chloride to form a final oil phase for emulsification.

Examples of organic solvents suitable for use within invention include, but are not limited to, methylene chloride(dichloromethane is the same thing as methylene chloride; methylene chloride is the common name people use most often, though dichloromethane is a better name for it), chloroform, ethyl acetate, or dimethyl sulfoxide. Methylene chloride and dimethyl sulfoxide are preferred.

The microspheres can be prepared by a variety of methods known in the art, including a single oil-in-water emulsion, a double oil-in-water emulsion, spray drying or coacervation of the polymer solution, for example (See, for example, U.S. Pat. Nos. 4,652,441; 4,711,782; 4,917,893; 5,061,492; 5,407,609; 5,478,564; 5,556,642; 5,631,021; 5,643,506; 5,945,126; 5,989,463; 6,224,794; 6,270,802; 6,403,114 and 6,90,700). Encapsulation performed via hydrophobic ion pairing offers the advantage of preparation of the microspheres by a single oil-in-water emulsion, which can offer improved release kinetics as well as ease of manufacturing, and may also improve core loading efficiencies and effect the form and distribution of the protein within the microsphere. Such microspheres may display desirable release kinetics, i.e., low initial burst and controlled release of the protein over time. HIP encapsulation allows for encapsulation of proteins of larger sizes (from 3 kD to over 60 kD), with the result that these encapsulated proteins have demonstrated effective release under physiological conditions. Such encapsulated proteins elicit strong and comprehensive immune responses, including both cellular and humoral immune responses. Protein antigens to be encapsulated into microspheres of the invention can also be of considerable length, including protein antigens of at least about 20 amino acid residues in length. In a preferred embodiment, the microspheres are formed by single emulsification of an oil phase containing the protein, an immunostimulant, and a wall-forming material into an aqueous phase using polyvinyl alcohol as a stabilizer. Alternatively, microspheres may be formed by a single emulsion technique without using HIP, such as by precipitating the protein out of aqueous solution by addition of excess ethanol, solubilization of the isolated precipitate with dimethyl sulfoxide, and then combining with methylene chloride and a wall-forming material to form a final oil phase for emulsification. HIP agents may also be added to the oil phase at this stage.

The microspheres of the invention preferably comprise a biodegradable polymer, such as poly(lactide-co-glycolide)(PLG), poly(lactide), poly(caprolactone), poly(hydroxybutyrate) and/or copolymers thereof. Alternatively, the microspheres can comprise another wall-forming material. Suitable wall-forming materials include, but are not limited to, poly(dienes) such as poly(butadiene) and the like; poly(alkenes) such as polyethylene, polypropylene, and the like; poly(acrylic) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers. Biodegradable microspheres (e.g., polylacide polyglycolide) for use as carriers are disclosed, for example, in U.S. Pat. Nos. 4,652,441; 4,711,782; 4,897,268; 4,917,893; 5,061,492; 5,075,109; 5,407,609; 5,476,663; 5,631,021; 5,811,128; 5,814,344; 5,820,883; 5,853,763; 5,928,647; 5,942,252; 6,024,938; 6,312,731 and 6,328,972.

In one embodiment, the polymer comprises PLG. The PLG can include ester end groups or carboxylic acid end groups, and have a molecular weight of from about 4 kDa to about 120 kDa, or preferably, about 8 kDa to about 65 kDa. Such polymers are commercially available, RG502H (PLG having carboxylic acid end groups) and RG503 (PLG having ester end groups), for example, are available from Boehringer Ingelheim GmbH, Ingelheim, Germany.

Typically, the method of the invention will result in the formation of microspheres of a suitable size for administration and delivery of proteins, particularly as vaccines Preferably, at least about 90% of the microspheres are about 1 to about 10 um in diameter.

The release rate of the microspheres will be influenced by the properties of the buffer used. In addition, the incorporation of fatty acid esters and cholesterol into microspheres to modify the release kinetics of encapsulated drug has been described by Urata et al., J. Controlled Release 58: 133-141, 1999, and these principles can be adapted for use with encapsulated proteins. Examples of fatty acid esters include, but are not limited to, ethyl myristate (C14), ethyl caprate (C10) and ethyl stearate (C18). Other excipients may also be co-encapsulated, such as poly(ethylene glycol)s, squalane, and squalene, which may also effect the release kinetics.

In certain embodiments, use of liposomes, nanocapsules, microparticles, lipid particles, vesicles, and the like, are also contemplated. In particular, the WT1 polypeptides or polynucleotides and immunostimulants of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol: 16:307-21, 1998; Takakura, Nippon Rinsho: 56:691-5, 1998; Chandran et al., Indian J Exp Biol.: 35:801-9, 1997; Margalit, Crit Rev Ther Drug Carrier Syst.: 12:233-61, 1995; U.S. Pat. Nos. 5,567,434; 5,552,157; 5,565,213; 5,738,868 and 5,795,587. Liposomes have been used successfully with a number of cell types that are normally difficult to transfect by other procedures, including T cell suspensions, primary hepatocyte cultures and PC 12 cells (Renneisen et al., J Biol Chem: 265:16337-42, 1990; Muller et al., DNA Cell Biol. (3):221-9, 1990). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for nanocapsule formulations. Nanocapsules can generally entrap compounds in a stable and reproducible way (see, for example, Quintanar-Guerrero et al., Drug Dev Ind Pharm. 24:1113-28, 1998). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 5:1-20, 1988; zur Muhlen et al., Eur J Pharm Biopharm. 45:149-55, 1998; Zambaux et al. J Controlled Release. 50:31-40, 1998; and U.S. Pat. No. 5,145,684.

Pharmaceutical Compositions

The pharmaceutical compositions of the invention comprise WT1 polypeptide or polynucleotide compositions in combination with immunostimulants either independently encapsulated or co-encapsulated or other combinations described herein, for use in prophylactic and therapeutic vaccine applications. Generally, such compositions will comprise one or more WT1 polypeptides or polynucleotides in combination with one or more immunostimulants co-encapsulated in to a microsphere formulation. Vaccine preparation is generally described in, for example, M. F. Powell and M. J. Newman, eds., “Vaccine Design (the subunit and adjuvant approach),” Plenum Press (NY, 1995). A strong and comprehensive immune response can be obtained to vaccine compositions comprising an antigen in combination with an adjuvant co-encapsulated in microspheres. Co-administration of encapsulated antigens and separately encapsulated adjuvant elicits a comprehensive immune response, and an even stronger comprehensive immune response can be obtained using antigen and adjuvant co-encapsulated in the same set of microspheres.

As used herein, pharmaceutically acceptable carrier includes any material which, when combined with an active ingredient, allows the ingredient to retain biological activity and is non-reactive with the subject's immune system. Examples include, but are not limited to, any of the standard pharmaceutical carriers such as a phosphate buffered saline solution, phosphate buffer, 5% dextrose in water, water, emulsions such as oil/water emulsion, and various types of wetting agents. Preferred diluents for aerosol or parenteral administration are phosphate buffered saline, normal (0.9%) saline, or 5% mannitol in water. Pharmaceutically acceptable carriers can further comprise any and all solvents, dispersion media, vehicles, coatings, diluents, antibacterial and antifungal agents, isotonic and absorption delaying agents, bulking agents (such as hydroxyethyl starch) wetting agents (such as Tween), antioxidants, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Pharmaceutical compositions as described herein may further include one or more excipients, preservatives, solubilizers, buffering agents, carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives, albumin to prevent protein loss on vial surfaces, etc. Methods of formulation are well known in the art and are disclosed, for example, in Remington: The Science and Practice of Pharmacy, Gennaro, ed., Mack Publishing Co., Easton Pa., 19^{th} ed., 1995.

The pharmaceutical compositions described herein may be presented in unit-dose or multi-dose containers, such as sealed ampoules or vials. Such containers are typically sealed in such a way to preserve the sterility and stability of the formulation until use. Therapeutic doses will generally be determined by the clinician according to accepted standards, taking into account the nature and severity of the condition to be treated, patient traits, etc. Determination of dose is within the level of ordinary skill in the art.

The development of suitable dosing and treatment regimens for using the particular compositions described herein in a variety of treatment regimens, including e.g., oral, parenteral, intravenous, intranasal, intradermal, sub-cutaneous and intramuscular administration and formulation, is well known in the art, some of which are briefly discussed below for general purposes of illustration. The amount of active compound contained within a sustained release formulation depends upon the site of implantation, the rate and expected duration of release and the nature of the condition to be treated or prevented.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature 386:410-4, 1997; Hwang et al., Crit Rev Ther Drug Carrier Syst 15:243-84, 1998; U.S. Pat. Nos. 5,641,515; 5,580,579 and 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 0.5% and 2% and about 60% or 70% or more of the weight or volume of the total formulation. In one embodiment the WT1 polypeptide is about 0.5% and the MPL® Adjuvant is about 0.05 to 0.5% by mass of the final lyophilized product. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, intradermally, sub-cutaneously or even intraperitoneally. Such approaches are well known to the skilled artisan, some of which are further described, for example, in U.S. Pat. Nos. 5,543,158; 5,641,515 and 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions, suspensions or dispersions and sterile powders for the extemporaneous preparation of sterile injectable solutions, suspensions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered and the liquid diluent isotonic. These particular aqueous solutions are especially suitable for intravenous, intramuscular, intradermal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. Nos. 5,756,353 and 5,804,212. Likewise, the delivery of drugs using intranasal microparticle resins (Takenaga et al., J Controlled Release 52:81-7, 1998) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

Therapy of Malignant Diseases

Immunologic approaches to cancer therapy are based on the recognition that cancer cells can often evade the body's defenses against aberrant or foreign cells and molecules, and that these defenses might be therapeutically stimulated to regain the lost ground, e.g. pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g. Jager, et al., Oncology 60:1-7, 2001; Renner, et al., Ann Hematol. 79:651-9, 2000.

Four basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8⁺ T cells. Polypeptide antigens that are selective or ideally specific for cancer cells, particularly cancer cells associated with WT1 expression, offer a powerful approach for inducing immune responses against cancer associated with WT1 expression, and are an important aspect of the present invention.

In further aspects of the present invention, the compositions and vaccines described herein may be used to inhibit the development of malignant diseases (e.g., progressive or metastatic diseases or diseases characterized by small tumor burden such as minimal residual disease). In general, such methods may be used to prevent, delay or treat a disease associated with WT1 expression. In other words, therapeutic methods provided herein may be used to treat an existing WT1-associated disease, or may be used to prevent or delay the onset of such a disease in a patient who is free of disease or who is afflicted with a disease that is not yet associated with WT1 expression.

As used herein, a disease is “associated with WT1 expression” if diseased cells (e.g., tumor cells) at some time during the course of the disease generate detectably higher levels of a WT1 polypeptide than normal cells of the same tissue. Association of WT1 expression with a malignant disease does not require that WT1 be present on a tumor. For example, overexpression of WT1 may be involved with initiation of a tumor, but the protein expression may subsequently be lost. Alternatively, a malignant disease that is not characterized by an increase in WT1 expression may, at a later time, progress to a disease that is characterized by increased WT1 expression. Accordingly, any malignant disease in which diseased cells formerly expressed, currently express or are expected to subsequently express increased levels of WT1 is considered to be “associated with WT1 expression.”

Immunotherapy may be performed using any of a variety of techniques, in which compounds or cells provided herein function to remove WT1-expressing cells from a patient. Such removal may take place as a result of enhancing or inducing an immune response in a patient specific for WT1 or a cell expressing WT1. Alternatively, WT1-expressing cells may be removed ex vivo (e.g., by treatment of autologous bone marrow, peripheral blood or a fraction of bone marrow or peripheral blood). Fractions of bone marrow or peripheral blood may be obtained using any standard technique in the art.

Within such methods, pharmaceutical compositions and vaccines may be administered to a patient. As used herein, a “patient” refers to any warm-blooded animal, preferably a human. A patient may or may not be afflicted with a malignant disease. Accordingly, the above pharmaceutical compositions and vaccines may be used to prevent the onset of a disease (i.e., prophylactically) or to treat a patient afflicted with a disease (e.g., to prevent or delay progression and/or metastasis of an existing disease). A patient afflicted with a disease may have a minimal residual disease (e.g., a low tumor burden in a leukemia patient in complete or partial remission or a cancer patient following reduction of the tumor burden after surgery radiotherapy and/or chemotherapy). Such a patient may be immunized to inhibit a relapse (i.e., prevent or delay the relapse, or decrease the severity of a relapse). Within certain preferred embodiments, the patient is afflicted with a leukemia (e.g., AML, CML, ALL or childhood ALL), a myelodysplastic syndrome (MDS); lymphoma or a cancer (e.g., gastrointestinal, ovarian, lung, thyroid or breast cancer or a melanoma), where the cancer or leukemia is WT1 positive (i.e., reacts detectably with an anti-WT1 antibody, as provided herein or expresses WT1 mRNA at a level detectable by RT-PCR, as described herein) or suffers from an autoimmune disease directed against WT1-expressing cells.

Other diseases associated with WT1 overexpression include kidney cancer (such as renal cell carcinoma, or Wilms tumor), as described in Satoh, et al., Pathol. Int. 50:458-71, 2000, and Campbell et al., Int. J. Cancer 78:182-8, 1998; and mesothelioma, as described in Amin, et al., Am. J. Pathol. 146:344-56, 1995. Harada et al., Mol. Urol. 3:357-364, 1999 describe WT1 gene expression in human testicular germ-cell tumors. Nonomura et al. Hinyokika Kiyo 45:593-7, 1999 describe molecular staging of testicular cancer using polymerase chain reaction of the testicular cancer-specific genes. Shimizu et al., Int. J. Gynecol. Pathol. 19:158-63, 2000 describe the immunohistochemical detection of the Wilms' tumor gene (WT1) in epithelial ovarian tumors.

WT1 overexpression was also described in desmoplastic small round cell tumors, by Barnoud, et al., Am. J. Surg. Pathol. 24:830-6, 2000; and Pathol. Res. Pract. 194:693-700, 1998. WT1 overexpression in glioblastoma and other cancer was described by Menssen, et al., J. Cancer Res. Clin. Oncol. 126:226-32, 2000, “Wilms' tumor gene (WT1) expression in lung cancer, colon cancer and glioblastoma cell lines compared to freshly isolated tumor specimens.” Other diseases showing WT1 overexpression include EBV associated diseases, such as Burkitt's lymphoma and nasopharyngeal cancer (Spinsanti et al., Leuk. Lymphoma 38:611-9, 2000, “Wilms' tumor gene expression by normal and malignant human B lymphocytes.”

In Leukemia 14:1634-4, 2000, Pan et al., describe in vitro IL-12 treatment of peripheral blood mononuclear cells from patients with leukemia or myelodysplastic syndromes, and reported an increase in cytotoxicity and reduction in WT1 gene expression. In Leukemia 13:891-900, 1999, Patmasiriwat et al. reported WT1 and GATA1 expression in myelodysplastic syndrome and acute leukemia. In Leukemia 13:393-9, 1999, Tamaki et al. reported that the Wilms' tumor gene WT1 is a good marker for diagnosis of disease progression of myelodysplastic syndromes. Expression of the Wilms' tumor gene WT1 in solid tumors, and its involvement in tumor cell growth, was discussed in relation to gastric cancer, colon cancer, lung cancer, breast cancer cell lines, germ cell tumor cell line, ovarian cancer, the uterine cancer, thyroid cancer cell line, hepatocellular carcinoma, in Oji et al., Jpn. J. Cancer Res. 90:194-204, 1999.

The encapsulated microsphere compositions provided herein may be used alone or in combination with conventional therapeutic regimens such as surgery, irradiation, chemotherapy and/or bone marrow transplantation (autologous, syngeneic, allogeneic or unrelated). As discussed in greater detail below, binding agents and T cells as provided herein may be used for purging of autologous stem cells. Such purging may be beneficial prior to, for example, bone marrow transplantation or transfusion of blood or components thereof. Binding agents, T cells, antigen presenting cells (APC) and compositions provided herein may further be used for expanding and stimulating (or priming) autologous, allogeneic, syngeneic or unrelated WT1-specific T-cells in vitro and/or in vivo. Such WT1-specific T cells may be used, for example, within donor lymphocyte infusions.

Routes and frequency of administration, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. In some tumors, pharmaceutical compositions or vaccines may be administered locally (by, for example, rectocoloscopy, gastroscopy, videoendoscopy, angiography or other methods known in the art). Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response that is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent complete or partial remissions, or longer disease-free and/or overall survival) in vaccinated patients as compared to non-vaccinated patients. In general, for pharmaceutical compositions and vaccines comprising one or more polypeptides, the amount of each polypeptide present in a dose ranges from about 100 μg to 5 mg. Suitable dose sizes will vary with the size of the patient, but will typically range from about 0.1 mL to about 5 mL. In one embodiment the ratio of WT1 polypeptide or polynucleotide to immunostimulant will be 1:1. In other embodiments the amount of one component can vary in relation to the other, for example the amount of the WT1 component or the immunostimulant may be 10 fold or greater than the other component.

In general, an appropriate dosage and treatment regimen provides the active compound(s) in an amount sufficient to provide therapeutic and/or prophylactic benefit. Such a response can be monitored by establishing an improved clinical outcome (e.g., more frequent complete or partial remissions, or longer disease-free and/or overall survival) in treated patients as compared to non-treated patients. Increases in preexisting immune responses to WT1 generally correlate with an improved clinical outcome. Such immune responses may generally be evaluated using standard proliferation, cytotoxicity or cytokine assays, which may be performed using samples obtained from a patient before and after treatment.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

The following Examples are offered by way of illustration and not by way of limitation.

### EXAMPLE 1

### Microspheres Containing Co-Encapsulated Protein and Adjuvant

AOT Precipitation

A protein-adjuvant microsphere formulation was prepared by a single emulsion method in which truncated TAT signal peptide fused to WT-1 F (SEQ ID NO:5) protein was precipitated using a hydrophobic ion pairing (HIP) technique. Three milligrams of protein in 8M urea, 90 mM sodium chloride, 50 mM sodium acetate, pH 5.5 was diluted with nanopure water to produce a final volume of 3.0 ml. To this was added 0.125 ml of 0.1 M CaCL₂ and 0.125 ml of 1.0 M HCl to lower the pH to approximately 3.0. The protein was precipitated by adding a 10 mg/mL aqueous AOT solution at a ratio of 2.3 mg AOT (docusate sodium) per 3 mg protein; equivalent to ˜2 AOT molecules per positive residue on the WT1 F fusion (at pH 3). The mixture was rocked at 24° C. for 45 minutes then centrifuged at 3000×g for 15 minutes to pellet the precipitated protein complex. The supernatant was poured off and the pelleted protein complex was then dissolved in 5 ml DMSO.

This WT-1/AOT/DMSO solution was then combined with a dichloromethane (DCM) solution containing 3 mg MPL® adjuvant (TEA salt, Corixa Corp., Seattle, Wash.), 300 mg PLG (RG502H Boehringer Ingelheim GmbH, Ingelheim, Germany), and 5 mL DCM. This formed a final oil phase that was a clear, single phase solution. This solution was emulsified in 400 mL 5% PVA (poly vinyl alcohol, 87-89% hydrolyzed, M_{w} 13-23 kDa, Sigma-Aldrich, St. Louis, Mo.) with a Silverson homogenizer (L4RT, Silverson Machines Inc., East Longmeadow, Mass.) at 9000 rpm for approximately 1.25 minutes. The microspheres were then hardened by stirring for 2-3 hours in an open container and then washed by centrifugation twice with water. Mannitol was added prior to freezing and lyophilization.

The resulting microspheres had a protein core loading of 1.1% and a protein core loading efficiency of 100%, as determined by amino acid analysis. The MPL core loading was 0.8% with core loading efficiency of 85% as determined by Bartlett assay (Bartlett and Lewis, Analytical Biochemistry 36, 159-167, 1970). Core loading is mass protein per mass microsphere (not including excipients which are not in the microspheres). The median particle size was 3 um as determined by laser light scattering particle size analysis (Horiba LA-920, Irvine, Calif.). The microspheres had a spherical morphology with low porosity as determined by scanning electron microscopy.

Protein release kinetics were monitored by the in vitro release of protein from microspheres at 37° C. in a release medium composed of 100 mM sodium phosphate, 8M urea buffer at pH 7.4 (urea was added to prevent precipitation of released protein). Controls included WT1 F fusion protein alone, WT1 F fusion protein plus placebo microspheres, and placebo microspheres alone. Samples were taken at 2 hours, 1, 2, 7, 14, 21, and 27 days. At each time point, the microspheres were centrifuged, an aliquot of supernant was collected, and fresh buffer was added to replace the removed supernant. Supernatant was analyzed by RP-HPLC using a diphenyl column (Vydac, Hesperia, Calif., Waters 2690 HPLC, Milford, Mass.). The microspheres had a low protein burst release, approximately 20% of protein released at 2 hours with no further significant release until 21 days with approximately 30% of protein released and further increase at 27 days.

Thus, WT-1 F-TAT fusion microsphere composition comprises the fusion protein and MPL encapsulated within poly(lactide-co-glycolide (PLG) microspheres. The protein was prepared by precipitating from buffer via hydrophobic ion pairing with AOT (docusate sodium) and then resuspending in dimethylsulfoxide (DMSO). The PLG has a 50:50 ratio of lactide and glycolide monomer, intrinsic viscosity around 0.2, with acid end groups. The microsphere had a low core loading of protein and MPL (about 1 wt % of each) and a small narrow particle distcibution (around 3-3.5 um median diameter).

Microspheres were also prepared using this AOT precipitation procedure with a different adjuvant, an aminoalkyl glucosaminide phosphate compound, RC529 (Corixa Corporation, Seattle, Wash.). The resulting protein/adjuvant co-encapsulated microspheres had the same properties as the protein/MPL® adjuvant co-encapsulated microspheres described above.

Ethanol Precipitation

A protein-adjuvant microsphere formulation was prepared by a single emulsion method in which truncated TAT signal peptide fused to WT-1 F (SEQ ID NO:5) protein was precipitated using an ethanol precipitation technique. Three milligrams of protein in 2.2 ml 8M urea, 90 mM sodium chloride, 50 mM sodium acetate, pH 5.5 was precipitated by adding six times the protein volume of absolute ethanol. The mixture was rocked at 4° C. for 45 minutes, then centrifuged at 1,000×g for 3 minutes to pellet the precipitated protein. The supernatant was poured off and the pelleted protein was then dissolved in 6.6 ml DMSO.

This DMSO solution containing the precipitated WT-1 fusion polypeptide was then combined with a DCM solution containing 10 mg AOT, 3 mg MPL® adjuvant (TEA salt, Corixa Corp., Seattle, Wash.) 300 mg PLG (RG503 Boehringer Ingelheim GmbH, Ingelheim, Germany), and 3.4 mL DCM. This formed a final oil phase that was a clear, single phase solution. This solution was emulsified in 280 mL 1.4% CMC (carboxymethyl cellulose sodium salt, DS 0.7, M_{w} ˜250,000, Sigma-Aldrich) with a Silverson homogenizer (L4RT, Silverson Machines Inc., East Longmeadow, Mass.) at 9000 rpm for 1.25 minutes. The microspheres were then hardened by stirring for 2-3 hours in an open container and then washed by centrifugation twice with water. Mannitol was added prior to freezing and lyophilization.

The resulting microsphere formulation had similar properties to those prepared using AOT precipitation. The protein/adjuvant co-encapsulated microspheres had a protein core loading of 1.0% and a protein core loading efficiency of 88%, as determined by amino acid analysis. The MPL core loading was 0.8% with core loading efficiency of 77% as determined by Bartlett assay. The median particle size was 3 um as determined by laser light scattering particle size analysis. The microspheres had a spherical morphology with low porosity as determined by scanning electron microscopy. The microspheres had a low protein burst release, approximately 18% protein released at 2 hours with no further significant release until 21 days with approximately 21% of protein released and further increase at 27 days.

### EXAMPLE 2

### CTL Responses 1N HLA-A2 Transgenic Mice with Immunized with Protein/Adjuvant Co-Encapsulated Microspheres

This protein/adjuvant co-encapsulated microspheres prepared above were characterized in an HLA-A2 transgenic mouse model for WT-1 immunogenicity.

Groups of four HLA-A2 Kb transgenic mice were immunized intradermally three times at three week intervals with each of the co-encapsulated microsphere formulations described above, at a dose of 15 μg protein and 1 μg adjuvant/mouse. Two weeks following the final immunization the mice were sacrificed and the spleens and sera were collected to assay for immune response.

Spleen cells were stimulated for one or two weeks in vitro with Jurkat DC-2.4-A2 Kb-WT-1-LAMP cells or Jurkat A2 Kb cells pulsed with WT-1 CD8 epitope peptides p10-18 (ALLPAVPSL), SEQ ID NO:7 or p37-45 (VLDFAPPGA), SEQ ID NO:8. A standard chromium release assay was used to assay for CTL activity. Target cells consisted of Jurkat A2 Kb cells pulsed with WT1 CD8 epitope peptide p10-18 (ALLPAVPSL), SEQ ID NO:7 or p37-45 (VLDFAPPGA), SEQ ID NO:8, Jurkat A2 Kb-WT-1-LAMP cells or Jurkat A2 Kb cells alone as non-specific controls.

CD4 cell activity was measured by in vitro stimulation of splenocytes for three days with 5 ug/ml rWT-1 fusion protein (SEQ ID NO:5), after which supernatants were assayed for secreted IFN-gamma by ELISA. Serum IgG₁ and IgG_{2b} antibody responses were assayed by ELISA.

Splenocytes from mice immunized with the WT-1 fusion polypeptide and MPL® adjuvant encapsulated microspheres prepared by the AOT precipitation method above exhibited a positive, WT-1 specific CTL response in 4/4 mice, a WT-1 specific IFN-γ CD4+ T cell response was also induced in all mice and high titers of WT-1 specific IgG1 and IgG2a were induced in all mice. The ratio of IgG2a:IgG1 was high, suggesting a Th1 type immune response was induced by immunization with this microsphere formulation.

Immunization with the WT-1 fusion polypeptide and MPL® adjuvant encapsulated microspheres prepared by the ethanol precipitation method above induced a Wt-1 specific positive CTL response in 2/4 mice, a WT-1 specific IFN-γ CD4+ T cell response was induced in 4/4 mice and high titers of WT-1 specific IgG1 and IgG2a were included in all mice. The ratio of IgG2a:IgG1 was high, suggesting a Th1 type immune response was induced by immunization with this microsphere formulation.

Immunization with the WT-1 fusion polypeptide and RC-529 encapsulated microspheres prepared by the AOT precipitation method above (having a 1:1 ratio of polypeptide to adjuvant) induced a WT-1 specific IFN-γ CD4+ T cell response and a high titer antibody with a high IgG1 and IgG2a ratio. CTL responses were negative from this formulation, but positive CD8+ T cell responses were induced by immunization with a similar formulation incorporating a lower dose of RC-529 (15 μg WT-1 truncated polypeptide and 1.5 μg RC-529).

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A composition comprising a WT-1 polypeptide of SEQ ID NO:5 and an immunostimulant co-encapsulated in biodegradable polymeric microspheres and a pharmaceutically acceptable carrier or excipient.

2. A composition according to claim 1, wherein the immunostimulants is an adjuvant.

3. A composition according to claim 2, wherein the adjuvant is selected from the group consisting of: monophosphoryl lipid A, 3-de-O-acylated monophosphoryl lipid A, an aminoalkyl glucosaminide 4-phosphate compound, a saponin, aluminum phosphate, calcium phosphate, cell wall skeleton, or a CpG-containing oligonucleotide, alone or in combination.

4. A composition according to claim 1, wherein the biodegradable polymeric microspheres comprise poly(lactide-co-glycolide) having ester end groups or carboxylic acid end groups, and a molecular weight of from about 4 kDa to about 120 kDa.

5. A composition according to claim 1, wherein the adjuvant is monophosphoryl lipid A or 3-de-O-acylated monophosphoryl lipid A; the WT1 polypeptide is a polypeptide comprising SEQ ID NO:5; and the biodegradable polymeric microspheres comprise poly(lactide-co-glycolide) having carboxylic acid end groups.

6. A method for enhancing or inducing an immune response in a human patient, comprising administering to a patient a composition comprising:
(a) immunostimulant and a WT-1 polypeptide of SEQ ID NO:5 co-encapsulated in biodegradable polymeric microspheres; and
(b) a physiologically acceptable carrier or excipient;
and thereby enhancing or inducing an immune response specific for WT1 antigen or a cell expressing WT1 antigen in the human patient.
